(19) United States
(12) Patent Application Publication
(10) Pub. No.: US 2011/0123445 A1
(43) Pub. Date: May 26, 2011

(54) TREATMENT MONITORING

(76) Inventor: Pamela Cohen, Tenafly, NJ (US)

(21) Appl. No.: 13/003,583

(22) PCT Filed: Jul. 13, 2009
(86) PCT No.: PCT/EP09/58935

§ 371 Date: Jan. 11, 2011

Related U.S. Application Data
(60) Provisional application No. 61/080,311, filed on Jul. 14, 2008.

(30) Foreign Application Priority Data
Aug. 12, 2008 (GB) 0814722.5

(51) Int. Cl. A61K 51/00; A61K 49/00

(52) U.S. Cl. 424/1.61; 424/9.1

(57) ABSTRACT

The present invention provides a method for monitoring the effectiveness of atreatment, wherein said treatment comprises an inhibitor designed to treat a disease comprising abnormal activity of the Ras/Raf/MEK/ERK pathway.

### TECHNICAL FIELD OF THE INVENTION

The present invention relates to in vivo imaging, and in particular to the application of in vivo imaging for monitoring treatment. Specifically, the present invention is suitable for the monitoring treatment of melanoma with agents that act on the Ras/Raf/MEK/ERK pathway.

### DESCRIPTION OF RELATED ART

A number of in vivo imaging agents based on RGD peptides are known to be suitable for detection of integrin expression, and are described for example in WO 2002/26776, WO 2003/006491, WO 2005/012335 and WO 2006/54904. These imaging agents target cell surface integrins and are taught as useful in the diagnosis of a range of angiogenesis-related disease states. These prior art references also teach the usefulness of these RGD peptide-based imaging agents in monitoring treatment of angiogenesis-related disease states.

Physiologically, the Ras/Raf/MEK/ERK signaling (MEK: Mitogen-activated protein kinase/Extracellular signal related kinase Kinase; ERK: Extracellular signal Related Kinase) pathway plays a central role in the regulation of a variety of cellular functions, including cellular proliferation, differentiation, survival, immortalization and angiogenesis (reviewed in Peyssonnaux and Eychene Biology of the Cell 2001; 93: 3-62). The pathway is highly conserved among all eukaryotes, and plays an integral role in the transduction of various extracellular signals into the nucleus. It is believed that the joint action of growth factors and integrins activates the Ras/Raf/MEK/ERK signalling pathway to result in angiogenesis (Hood et al J. Cell Biol. 2003; 162(5): 933-43). This is supported by the fact that inhibition of αᵥβ₃-integrin expression suppresses ERK signalling leading to endothelial apoptosis and inhibition of angiogenesis (Eliceiri et al J Cell Biol. 1998; 140: 1255-1263). In addition to this “outside-in” signalling, a parallel “inside-out” signalling mechanism has been shown. Woods et al (Mol. Cell Biol. 2001; 21(9): 3192-205) demonstrated that pharmacological inhibition of MEK1 in human melanoma cell lines led to a reduced expression of cell surface α₆ and β₃ integrins.

Aberrant or inappropriate function of the Ras/Raf/MEK/ERK pathway has been identified in a range of diseases, in particular the constitutive activation of the Ras/Raf/MEK/ERK pathway in the oncogenic behavior of cancers. Activating mutations of Ras are found in around 30% of all cancers (Bos Cancer Research 1989; 49(17): 4682-9), including in 9-15% of melanomas. B-Raf somatic missense mutations conferring constitutive activation are more frequent in melanoma and found in 60-66% malignant cutaneous melanomas (Davies et al., Nature 2002; 417: 949 954). The most frequent mutation in B-Raf (80%) is a glutamic acid for valine substitution at position 599 (V599E). These B-raf mutations increase the basal kinase activity of B-Raf and are thought to uncouple Ras/Raf/MEK/ERK signalling from upstream proliferation drives including Ras and growth factor receptor activation, resulting in constitutive activation of ERK. Mutated B-Raf proteins have been demonstrated to be essential for melanoma cell viability and transformation (Hingorani et al., Cancer Res. 2003; 63: 5198-5202).

Over the last few years, a number of abstracts and scientific papers have published relating to the evaluation for cancer treatment of compounds that inhibit B-Raf (Eisen et al Br. J. Cancer 2006; 95P: 581-6; Gatzemeier et al Proc. Am. Soc. Clin. Oncol. 2006; 24: abstract 7002; Wu et al Prostate Cancer Symposium. San Francisco, Calif., USA. Feb. 24-26, 2006: abstract 259; and, Flaherty et al Proc. Am. Soc. Clin. Oncol. 2003; 22: 410). In addition, there have been various publications relating to the evaluation for cancer treatment of compounds that inhibit MEK (Sebolt-Leopold et al Nat. Med. 1999; 5: 810-6; Sebolt-Leopold et al Nat. Rev. Cancer 2004; 4: 937-47; Alessi et al J. Biol Chem. 1995; 270(46): 27489-94; Haas et al Melanoma Res. 2006; 16: S92-S93; Adjei et al J. Clin. Oncol. 2008; 26(13): 2139-46; and, Mackin et al J. Surgical Res. 2006; 130(2): 262).

Inhibition of B-Raf or MEK would be expected to effectively treat melanomas having activating mutations of Ras or B-Raf. However, a proportion of melanomas would not respond to such treatment. In order to select the best treatment regimen, it would be valuable to be able to distinguish between melanomas that are either (i) responsive or (ii) not responsive to B-Raf or MEK inhibitors.

### SUMMARY OF THE INVENTION

The present invention provides a method that overcomes the above-described problems of the prior art. The method of the invention uses in vivo imaging to monitor the effectiveness of melanoma treatments, and as such is minimally invasive. The method of the invention can be used in observing early response to a treatment, and can help to decide whether a particular treatment is suitable, or should be continued. As well as being useful in monitoring the effectiveness of treatments approved for clinical use, the method of the invention finds application in the development of new treatments, for example in the determination of optimal doses and regimens, and for identifying subjects most likely to respond to treatment.

### DETAILED DESCRIPTION OF THE INVENTION

In one aspect, the present invention provides a method for monitoring the effectiveness of an inhibitor, wherein said inhibitor is an inhibitor of B-raf, MEK1/2 (MEK: Mitogen-activated protein kinase/Extracellular signal related kinase Kinase), or ERK1/2 (ERK: Extracellular signal Related Kinase), said inhibitor being used to treat a subject suffering from melanoma, said method comprising:
(a) at a first point in time, carrying out an in vivo imaging procedure on said subject to generate a first in vivo image of a region of interest, wherein said region of interest comprises said melanoma, and wherein said in vivo imaging procedure comprises:
   (i) administration to said subject of an in vivo imaging agent comprising a vector labelled with an in vivo imaging moiety, wherein said in vivo imaging agent binds to αᵥβ₃ integrin with a Ki of <10 nM in a competitive binding assay for αᵥβ₃ integrin where the Ki value is determined by competition with echistatin;
   (ii) allowing the administered in vivo imaging agent of step (i) to bind to αᵥβ₃ integrin expressed by said melanoma;
   (iii) detecting signals emitted by the in vivo imaging moiety of the bound in vivo imaging agent of step (ii); and,
   (iv) converting the signals detected in step (iii) into a first in vivo image representative of αᵥβ₃ integrin expression on the surface of said melanoma cells in said region of interest;
(b) treating said subject with said inhibitor;
(c) at a second point in time, repeating the in vivo imaging procedure as defined in step (a) to generate a second in vivo image of said region of interest; and,
(d) comparing said first in vivo image with said second in vivo image, whereby a decrease in said detected signals at said second point in time indicates effectiveness of said inhibitor in treating said melanoma.

The “subject” of the invention can be any human or animal subject. Preferably the subject of the invention is a mammal. Most preferably, said subject is an intact mammalian body in vivo. In an especially preferred embodiment, the subject of the invention is a human.

In the context of the present invention, the term “Ras/Raf/MEK/ERK pathway” takes the meaning known in the art, i.e. a signal transduction pathway comprising a chain of protein kinases which ultimately act to regulate the activity of transcription factors. In response to a variety of signals including growth factors, cytokines, UV radiation, and stress-inducing agents, Raf family members are recruited to the plasma membrane upon binding to guanosine triphosphate (GTP) loaded Ras, resulting in the phosphorylation and activation of Raf proteins. Activated Raf proteins then phosphorylate and activate MAPK/ERK kinase 1/2 (MEK1/2), which in turn phosphorylate and activate extracellular signal regulated kinase 1/2 (ERK 1/2). Upon activation, ERKs translocate from the cytoplasm to the nucleus resulting in the phosphorylation and regulation of activity of transcription factors such as Elk-I and Myc. Aberrant activity of the Ras/Raf/MEK/ERK pathway is associated with tumour metastasis.

An “inhibitor of B-raf, MAPK/ERK kinase 1/2 (MEK1/2), or extracellular signal regulated kinase 1/2 (ERK1/2)” is a compound that targets the Ras/Raf/MEK/ERK pathway by inhibition of B-raf, MEK1/2 or ERK1/2, respectively. MEK1/2 and ERK1/2 are protein kinases downstream of B-Raf in the Ras/Raf/MEK/ERK pathway. The step of “treating” said subject with said inhibitor is carried out by administration of said inhibitor to said subject in a suitable pharmaceutical composition. Broadly speaking, a “pharmaceutical composition” comprises an active agent together with a biocompatible carrier, in a form suitable for mammalian administration. Where said active agent is an inhibitor of B-raf, MEK1/2 or ERK1/2, the “biocompatible carrier” is a pharmaceutically acceptable vehicle in which the active agent is incorporated, such that the composition is physiologically tolerable, i.e. can be administered to the mammalian body without toxicity or undue discomfort. The choice of biocompatible carrier depends on the route of administration. Inhibitors of the method of the present invention may be administered orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents.

The amount of inhibitor that may be combined with the carrier materials to produce a pharmaceutical composition in a single dosage form will vary depending upon the host treated, and the particular mode of administration. Preferably, the inhibitor compositions should be formulated so that a dosage of between 0.01-100 mg/kg body weight/day of the inhibitor can be administered to a subject. It should also be understood that a specific dosage and treatment regimen for any particular subject will depend upon a variety of factors, including the activity of the specific inhibitor employed, the age, body weight, general health, sex, diet, time of application, rate of excretion, drug combination, and the judgment of the treating physician and the severity of the particular disease being treated. The amount of an inhibitor in the composition will also depend upon the particular inhibitor.

A preferred inhibitor is selected from agents that inhibit (i) B-Raf, such as BAY 43-9006, PLX 4032 and CHIR-265; and (ii) MEK, such as PD184352, PD098059, PD0325901, AZD6244, and U0126. Each of these is now described in further detail.

BAY 43-9006 (sorafenib/Nexavar®, Bayer Pharmaceuticals) is a multikinase inhibitor with a broad spectrum of antitumour activity on cancer-cell proliferation and angiogenesis (Wilhelm et al Cancer Res. 2004; 64: 7099-7109). One of its targets is B-Raf. The efficacy of BAY 43-9006 in non-small-cell lung cancer, prostate cancer, and melanoma has undergone assessment in a randomised phase II trial (Eisen et al Br. J. Cancer 2006; 95P: 581-6) and in single-arm phase II clinical studies (Gatzemeier et al Proc. Am. Soc. Clin. Oncol. 2006; 24: abstract 7002; and Wu et al Prostate Cancer Symposium. San Francisco, Calif., USA. Feb. 24-26, 2006: abstract 259). Other medications are being developed that may have higher affinity and specificity for B-Raf inhibition compared with BAY 43-9006 are PLX 4032 and CHIR-265, both of which have been, or are being, evaluated in Phase I trials (Flaherty et al Proc. Am. Soc. Clin. Oncol. 2003; 22: 410; and www.plexxicon.com).

PD184352 (CI-1040, Pfizer) is an oral, selective small-molecule inhibitor of MEK 1/2. PD184352 was the first MEK inhibitor reported to inhibit tumour growth in vivo (Sebolt-Leopold et al Nat. Med. 1999; 5: 810-6; and Sebolt-Leopold et al Nat. Rev. Cancer 2004; 4: 937-47). PD0325901 (Pfizer), a second generation MEK inhibitor entered clinical development and appears to have good pharmacologic properties, which investigators hope may translate into better anti-cancer efficacy. In phase II studies biopsied tumour tissue demonstrated phosphorylated ERK suppression at all dose levels and in all tumour types, including melanoma, breast, colon, and lung.

PD098059 blocks the Ras/Raf/MEK/ERK pathway by inhibiting activation of MEK 1/2 by Raf and does not inhibit the phosphorylation of other Raf or MEK kinase substrates, indicating that it exerts its effect by binding to the inactive form of MEK 1/2. PD098059 also acts as a specific inhibitor of the activation of MEK in Swiss 3T3 cells, suppressing by 80-90% its activation by a variety of agonists (Alessi et al J. Biol. Chem. 1995; 270(46): 27489-94).

AZD6244 (Astra Zeneca) is a MEK inhibitor that has been shown to block the growth of B-Raf V600E-bearing melanoma in vitro and in vivo (Haas et al Melanoma Res. 2006; 16: S92-S93). A phase I trial in a group of 57 cancer patients was documented reporting that AZD6244 is well tolerated with demonstrable inhibition of ERK phosphorylation at the recommended phase II dose (Adjei et al J. Clin. Oncol. 2008; 26(13): 2139-46). Recently, Astra Zeneca and Merck announced a collaboration to research a treatment comprising AZD6244 and MK-2206 (www.astrazeneca.com). MK-2206 interacts with the phosphatidylinositol-3 kinase pathway.

UO126 blocks the Ras/Raf/MEK/ERK pathway by inhibition of MEK. The cancer cell lines HepG2 (hepatoma), Ht-29 (colon), MiaPaca (pancreas) and Panc-1 (pancreas) were treated with UO126 for 45 minutes at concentrations ranging from 20-50 uM, and was shown to have considerable activity in the cell lines tested (Mackin et al J. Surgical Res. 2006; 130(2): 262).

“Monitoring the effectiveness” of any one of such inhibitors comprises selection of a measurable characteristic of the diseased tissue the inhibitor seeks to treat. In the case of the present invention, the measurable characteristic is activity of the Ras/Raf/MEK/ERK pathway, which is inferred from the level of expression of αᵥβ₃ integrin.

In order for monitoring to be of value, particular time points are selected where, if the inhibitor is effective in treating the diseased tissue, a reduction in the activity of the Ras/Raf/MEK/ERK pathway in the cells of the diseased tissue would be anticipated. For example, the “first point in time” can usefully be following a diagnosis that the subject is suffering from melanoma, and before the inhibitor is applied to the subject. A “second point in time” is selected after treatment with the inhibitor has been commenced. Where the time needed for the inhibitor to take effect is known, this second point in time can be chosen to be at around this time. In other cases, especially where a new drug candidate is being evaluated, this time may not be known so that a second point in time needs to be empirically determined. Where activity of the Ras/Raf/MEK/ERK pathway decreases over time, this is an indication that the inhibitor has been effective. In a preferred embodiment of the method of the invention, further points in time may be selected to monitor the progress of treatment with the inhibitor throughout the entire treatment period, up to a defined end point such as remission of the disease.

Melanomas comprising a mutation in the B-Raf gene would be anticipated to respond well to inhibitors of B-Raf, MEK1/2 or ERK1/2. Therefore, a preferred melanoma in the method of the invention is melanoma comprising a mutation in the B-raf gene, in particular the mutation resulting from V599E substitution.

The term “in vivo imaging” as used herein refers to those techniques that noninvasively produce images of all or part of the internal aspect of the subject of the invention. Preferred in vivo imaging methods for use in the present invention are single photon emission computed tomography (SPECT), positron emission tomography (PET), and magnetic resonance imaging (MRI). Most preferred in vivo imaging methods are SPECT or PET, with PET being especially preferred. The preference for PET in the method of the invention is due to its excellent sensitivity and resolution, so that even relatively small changes in a lesion can be observed over time. PET scanners routinely measure radioactivity concentrations in the picomolar range. Micro-PET scanners now approach a spatial resolution of about 1 mm, and clinical scanners about 4-5 mm.

The in vivo imaging method of the present invention begins with “administration” to said subject of an in vivo imaging agent, wherein said in vivo imaging agent comprises a vector labelled with an in vivo imaging moiety. Parenteral administration is preferred for the administration of an in vivo imaging agent, most preferably intravascular administration. Following administration, the in vivo imaging agent is allowed to bind to αᵥβ₃ integrin expressed by said melanoma. The in vivo imaging agent is selected such that it binds to αᵥβ₃ integrin with a Ki of <10 nM, preferably <5 nM. To determine Ki, a known competitive binding assay for αᵥβ₃ integrin can be used where the Ki value is determined by competition with echistatin (Kumar et al J Pharmacol Exp Ther. 1997; 283: 843-853).

The term “labelled with an in vivo imaging moiety” means that the in vivo imaging moiety is either a constitutive part of the vector, e.g. a ¹¹C, ¹⁸F atom. Alternatively, the in vivo imaging moiety forms part of a chemical group that is conjugated to the vector, and an optional linker moiety links the vector and the in vivo imaging moiety together. The “in vivo imaging moiety” comprises an atom that emits signals that may be detected externally to said subject following administration. Preferred in vivo imaging moieties of the invention are described below.

The “vector” is a chemical compound that binds αᵥβ₃ integrin. As discussed in the prior art section, such compounds include RGD-containing peptides and corresponding peptidomimetics, as well as monoclonal antibodies. By definition, echistatin and derivatives thereof that bind to αᵥβ₃ integrin are also suitable vectors. A “peptidomimetic” is a compound containing non-peptidic structural elements that is capable of mimicking or antagonising the biological actions of a natural parent peptide. Methods to obtain in vivo imaging agents comprising such vectors are described in the art (see for example: Hua et al Circulation 2005; 111: 3255-3260; Bach-Gansmo et al J Nucl Med 2006; 47: 1434-1439; Sipkins et al Nature Medicine 1998; 4(5): 623-6; Ellegala et al (Circulation 2003; 108: 336-41; Winter et al, Circulation 2003; 108: 2270-4). Suitably, when the vector is an RGD-containing peptide, it is between 5 and 20 amino acids long, preferably between 6 and 12, most preferably between 8 and 10. Some preferred RGD peptides are described in more detail below.

A “linker moiety” of the present invention is a bivalent radical of Formula -(L)ₙ- wherein:
each L is independently —C(═O)—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₄₋₈ cycloheteroalkylene group, a C₄₋₈ cycloalkylene group, a C₅₋₁₂ arylene group, a C₃₋₁₂ heteroarylene group, an amino acid, a polyalkyleneglycol, polylactic acid or polyglycolic acid moiety;
n is an integer of value 1 to 15;
each R′ group is independently H or C₁₋₁₀ alkyl, C₃₋₁₀ alkylaryl, C₂₋₁₀ alkoxyalkyl, C₁₋₁₀ hydroxyalkyl, C₁₋₁₀-fluoroalkyl, or 2 or more R′ groups, together with the atoms to which they are attached form a carbocyclic, heterocyclic, saturated or unsaturated ring.

Preferably, said linker moiety is a chain of between 10 and 100 atoms, most preferably between 10 and 50 atoms. Preferred L groups are —C(═O)—, —CH₂—, —NH—, —NHC(═O)—, —C(═O)NH—, —CH₂—O—CH₂—, and amino acids. Specifically excluded are linker moieties wherein 2 or more carbonyl groups are linked together, or wherein 2 or more heteroatoms are linked together. The skilled person would understand that these are either not chemically feasible, or are too reactive or unstable to be suitable for use in the present invention.

A preferred in vivo imaging moiety is selected from:
(a) a radioactive metal ion;
(b) a gamma-emitting radioactive halogen;
(c) a positron-emitting radioactive non-metal;
(d) a paramagnetic metal ion.

In vivo imaging moieties (a)-(c) are preferred, with (c) being most preferred.

The “detecting” step of the method of the invention involves the of signals emitted by the in vivo imaging moiety of the in vivo imaging agent by means of a detector sensitive to said signals. Examples of signals emitted by the in vivo imaging moiety that are suitable for use in the present invention are those that may be detected externally to said subject following administration.

The “converting” step of the method of the invention is carried out by a computer which applies a reconstruction algorithm to the detected signals to yield a dataset. This dataset is typically manipulated to generate said first and second in vivo images, illustrating areas within the subject representative of αᵥβ₃ integrin expression on the surface of cells. The dataset may also be evaluated before/without generation of any in vivo image to observe changes over time in the quantity and intensity of the detected signals. However, production of in vivo images is preferred as more information can be obtained, e.g. the location and size of the diseased tissue.

Due to the known association between activation of the Ras/Raf/MEK/ERK pathway and αᵥβ₃ integrin expression (Woods et al Molecular and Cellular Biology 2001; 21(9): 3192-3205), the present inventors propose that αᵥβ₃ integrin expression can be used as a surrogate marker of Ras/Raf/MEK/ERK pathway activity.

In a preferred embodiment, the vector of the in vivo imaging agent is an RGD peptide. A number of such in vivo imaging agents are known in the art, and are taught to be useful for in vivo imaging of αᵥβ₃ integrin expression (see for example Zhang et al Cancer Res. 2007; 67(4): 1555-62, Beer et al Clin. Cancer Res. 2006; 12(13): 3942-9, WO 02/26776, WO 2003/006491, WO 2005/12335 and WO 2005/123767).

A preferred RGD peptide-based in vivo imaging agent for use in the method of the invention is of Formula I:

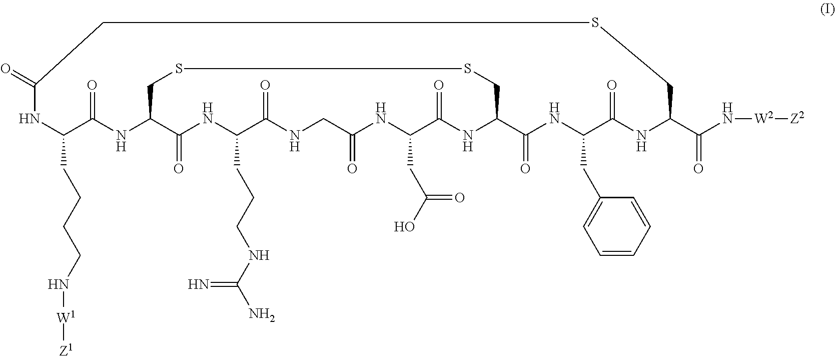

wherein:
W₁ and W₂ are independently an optional linker moiety, wherein said linker moiety is as defined above; and,
Z₁ and Z₂ are independently (i) a group comprising an in vivo imaging moiety, (ii) a sugar moiety, or (iii) hydrogen, with the proviso that at least one of Z₁ and Z₂ is an in vivo imaging moiety.

The peptide part of the in vivo imaging agent of Formula I can be synthesised using all known methods of chemical synthesis but particularly useful is the solid-phase methodology of Merrifield employing an automated peptide synthesiser (J. Am. Chem. Soc. 1964; 85: 2149). Standard procedures for the synthesis strategy are described in E. Atherton & R. C. Sheppard, “Solid phase peptide synthesis: a practical approach, 1989, IRL Press, Oxford.

A synthesis resin with an acid-labile linker group, to which the desired protected C-terminal amino acid residue is attached by amide bond formation, is used. For example, a so-called Rink amide AM resin with a (dimethoxyphenyl-aminomethyl)-phenoxy-derived linker may be applied (Rink, Tetrahedron Lett. 1987; 30: 3787). Acidolytic cleavage of the peptide from this resin will yield a peptide amide. Alternatively, a O-Bis-(aminoetyl)ethylene glycol trityl resin (Barlos et al, Liebigs Ann. Chem. 1988; 1079) can be used that upon acidolytic cleavage yields a peptide with a primary amine handle.

Labelling the vector with an in vivo imaging moiety to obtain the in vivo imaging agent may be conveniently carried out by means of a “precursor compound”, which is a derivative of the vector that targets αᵥβ₃ expression, designed so that chemical reaction with a convenient chemical form of the desired in vivo imaging moiety/moieties occurs site-specifically; can be conducted in a minimal number of steps (ideally a single step); and without the need for significant purification (ideally no further purification), to give the desired in vivo imaging agent. Such precursor compounds are synthetic and can conveniently be obtained in good chemical purity.

The precursor compound may be provided as part of a kit, which is particularly convenient for the preparation of radiolabelled in vivo imaging agents in radiopharmacies. Such a kit may contain a cartridge which can be plugged into a suitably adapted automated synthesiser. The cartridge may contain, apart from the precursor compound, a column to remove any unwanted radioactive ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customers' requirements for radioactive concentration, volumes, time of delivery, etc. Conveniently, all components of the kit are disposable to minimise the possibility of contamination between runs, and also may be sterile and quality assured.

The precursor compound may optionally comprise one or more protecting groups for certain functional groups of the vector. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferably, when present, the linker moiety acts as a biomodifier moiety. A “biomodifier moiety” has the function of modifying the pharmacokinetics and blood clearance rates of the in vivo imaging agent. An example of a suitable biomodifier moiety is one based on a monodisperse PEG building block comprising 1 to 10 units of said building block. Additionally, said biomodifier moiety may also represent 1 to 10 amino acid residues. Preferred amino acid residues for said biomodifier moiety are charged amino acids such as lysine and glutamic acid, or charged non-natural amino acids such as cysteic acid and phosphonoalanine. In addition, the amino acids glycine, aspartic acid and serine may be included. In a preferred embodiment, the biomodifier moiety comprises a monodisperse PEG-like structure, the 17-amino-5-oxo-6-aza-3,9,12,15-tetraoxaheptadecanoic acid of Formula II:

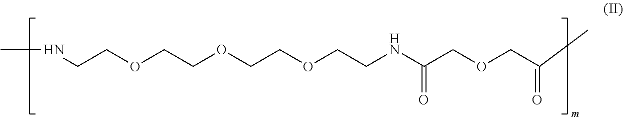

wherein m equals an integer from 1 to 10 and where the C-terminal unit is an amide moiety. The biomodifier moiety acts to modify the pharmacokinetics and blood clearance rates of the in vivo imaging agents. The function of the biomodifier moiety in the present invention is to decrease uptake in the tissues and increase excretion via the kidneys, thereby resulting in less background interference and giving a better in vivo image. The biomodifier moiety can further represent a moiety preferentially derived from glutaric and/or succinic acid and/or a polyethyleneglycol based unit and/or a unit of Formula II as illustrated above. For the purposes of the present invention, one of the primary aims of the biomodifier moiety is to reduce the background tissue uptake of the in vivo imaging agent. This ensures optimal detection of the signal emitted from specifically-bound in vivo imaging agent relative to background signal. The nature of the linker moiety should not interfere with the affinity of the in vivo imaging agent for its target receptors. In addition, the linker moiety should not act to increase the background liver uptake of the in vivo imaging agent, such as may occur if e.g. an overly-large polyethyleneglycol based unit were to be used.

Where either Z₁ or Z₂ is a sugar moiety it too may act as a biomodifier moiety as defined above. A “sugar moiety” is a carbohydrate group which is usually an aldehyde or a ketone derivative of a polyhydric alcohol. It may be a monomer (monosaccharide), such as fructose or glucose, or two sugars joined together to form a disaccharide. Disaccharides include sugars such as sucrose, which is made of glucose and fructose. The term sugar includes both substituted and non-substituted sugars, and derivatives of sugars. Preferably, the sugar is selected from glucose, glucosamine, galactose, galactosamine, mannose, lactose, fucose and derivatives thereof, such as sialic acid, a derivative of glucosamine. The sugar is preferably α or β. The sugar may especially be a manno- or galactose pyranoside. The hydroxyl groups on the sugar may be protected with, for example, one or more acetyl groups. The sugar moiety is preferably N-acetylated. Preferred examples of such sugars include N-acetyl galactosamine, sialic acid, neuraminic acid, N-acetyl galactose, and N-acetyl glucosamine.

It has been shown recently that sugar conjugation can favourably alter pharmacokinetics of in vivo imaging agents. Carbohydration leads to reduced lipophilicity of small radiolabelled peptides and, thus, to a dramatic reduction of hepatobiliary in favour of renal excretion (Haubner et al J. Nuc. Med. 2001; 42: 326-36).

When the in vivo imaging moiety comprises a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; or γ-emitters such as ^{99m}Tc, ¹¹¹In, ^{113m}In, or ⁶⁷Ga.

Preferred radiometals are ^{99m}Tc, ⁶⁴Cu, ⁶⁸Ga and ¹¹¹In. Most preferred radiometals are γ-emitters, especially ^{99m}Tc.

When the in vivo imaging moiety comprises a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the in vivo imaging moiety comprises a metal ion, it is preferably present as a metal complex of the metal ion with a synthetic ligand. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include other excipients in the preparation in vitro (e.g. radioprotectants or antimicrobial preservatives used in the preparation), or endogenous compounds in vivo (e.g. glutathione, transferrin or plasma proteins). The term “synthetic” has its conventional meaning, i.e. man-made as opposed to being isolated from natural sources e.g. from the mammalian body. Such compounds have the advantage that their manufacture and impurity profile can be fully controlled.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as MIBI (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

When the metal ion is technetium, suitable chelating agents which form metal complexes resistant to transchelation include, but are not limited to:

(i) diaminedioximes;
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam dioxocyclam; and,
(v) N₂O₂ ligands having a diaminediphenol donor set.

Preferred chelating agents of the invention when the in vivo imaging moiety comprises technetium are diaminedioximes and tetraamines. The structure of, and a method to obtain, a preferred diaminedioxime chelating agent are disclosed in WO 03/006070. The structure of, and a method to obtain, a preferred tetraamine chelating agent are disclosed in WO 06/008496.

The above described ligands are particularly suitable for complexing technetium e.g. ^{94m}Tc or ^{99m}Tc, and are described more fully by Jurisson et al (Chem. Rev. 1999; 99: 2205-2218). The ligands are also useful for other metals, such as copper (⁶⁴Cu or ⁶⁷Cu), vanadium (e.g. ⁴⁸V), iron (e.g. ⁵²Fe), or cobalt (e.g. ⁵⁵CO).

Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. Particularly preferred for gadolinium are chelates including DTPA, ethylene diamine tetraacetic acid (EDTA), triethylene tetraamine hexaacetic acid (TTHA), 1,4,7,10-tetraazacyclododecane-1,4,7,10-tetraacetic acid (DOTA), 10-(2-hydroxypropyl)-1,4,7,10-tetraazacyclododecane-1,4,7-triacetic acid (DO3A) and derivatives of these.

When the in vivo imaging moiety is part of a metal complex, an associated linker moiety, as previously defined herein, is preferably present. The role of the linker moiety in this case is to distance the relatively metal complex, which results upon metal coordination, from the active site of the peptide so that e.g. substrate binding is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site. Preferred linker moieties in the context of these chelators have a backbone chain which contains 2 to 10 atoms, most preferably 2 to 5 atoms, with 2 or 3 atoms being especially preferred. A minimum linker group backbone chain of 2 atoms confers the advantage that the chelator is well-separated from the peptide so that any interaction is minimised. Furthermore, the peptide is unlikely to compete effectively with the coordination of the chelator to the metal ion. In this way, both the biological targeting characteristics of the peptide, and the metal complexing capability of the chelator are maintained. It is strongly preferred that the metal complex is bound to the peptide in such a way that the linkage does not undergo facile metabolism in blood. That is because such metabolism would result in the imaging metal complex being cleaved off before the in vivo imaging agent reaches the desired in vivo target site. The peptide is therefore preferably covalently bound to the metal complex via linker moieties comprising linkages which are not readily metabolised. Suitable such linkages are carbon-carbon bonds, amide bonds, urea or thiourea linkages, or ether bonds.

Non-peptide linker groups such as alkylene groups or arylene groups have the advantage that there is no significant hydrogen bonding with the peptide part of Formula I so that the linker does not interact with the peptide. Preferred alkylene spacer groups are —(CH₂)_{q}— where q is an integer of value 2 to 5. Preferably q is 2 or 3. Preferred arylene spacers are of Formula III:

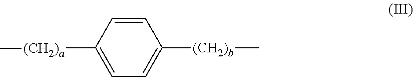

where: a and b are each independently 0, 1 or 2.

A preferred linker moiety here is thus —CH₂CH₂-(L)ₚ- where L is as defined above and p is an integer of value 0 to 3. Most preferably, -(L)ₚ- is —CO— or —NR—.

When the imaging metal is technetium, the usual technetium starting material is pertechnetate, i.e. TcO₄⁻ which is technetium in the Tc(VII) oxidation state. Pertechnetate itself does not readily form metal complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

When the in vivo imaging moiety is a gamma-emitting radioactive halogen, the radiohalogen is suitably chosen from ¹²³I, ¹³¹I or ⁷⁷Br. ¹²⁵I is specifically excluded as it is not suitable for use as an in vivo imaging moiety for external in vivo imaging. A preferred gamma-emitting radioactive halogen for in vivo imaging is ¹²³I.

Where the in vivo imaging moiety is radioiodine, the in vivo imaging agent of Formula I can be obtained by means of a precursor compound comprising a derivative which either undergoes electrophilic or nucleophilic iodination or undergoes condensation with a labelled aldehyde or ketone. Examples of the first category are:
(a) organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates);
(b) a non-radioactive alkyl bromide for halogen exchange or alkyl tosylate, mesylate or triflate for nucleophilic iodination;
(c) aromatic rings activated towards nucleophilic iodination (e.g. aryl iodonium salt aryl diazonium, aryl trialkylammonium salts or nitroaryl derivatives).

Preferred such precursor compounds comprise: a non-radioactive halogen atom such as an aryl iodide or bromide (to permit radioiodine exchange); an organometallic precursor compound (e.g. trialkyltin, trialkylsilyl or organoboron compound); or an organic precursor such as triazenes or a good leaving group for nucleophilic substitution such as an iodonium salt. Preferably for radioiodination, the precursor compound comprises an organometallic precursor compound, most preferably trialkyltin.

Precursor compounds and methods of introducing radioiodine into organic molecules are described by Bolton (J. Lab. Comp. Radiopharm. 2002; 45: 485-528). Suitable boronate ester organoboron compounds and their preparation are described by Kabalka et al (Nucl. Med. Biol. 2002; 29: 841-843, and Nucl. Med. Biol. 2003; 30: 369-373). Suitable organotrifluoroborates and their preparation are described by Kabalka et al (Nucl. Med. Biol. 2004; 31: 935-938).

Examples of aryl groups to which radioactive iodine can be attached are given below:

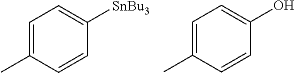

Both contain substituents which permit facile radioiodine substitution onto the aromatic ring. A tyrosine residue permits radioiodination to be carried out using its inherent phenol group.

Alternative substituents containing radioactive iodine can be synthesised by direct iodination via radiohalogen exchange, e.g.

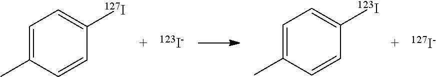

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the in vivo imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

Radiofluorination may be carried out via direct labelling using the reaction of ¹⁸F-fluoride with a suitable chemical group in a precursor compound having a good leaving group, such as an alkyl bromide, alkyl mesylate or alkyl tosylate. ¹⁸F can also be introduced by alkylation of N-haloacetyl groups with a ¹⁸F(CH₂)₃OH reactant, to give —NH(CO)CH₂O(CH₂)₃¹⁸F derivatives. For aryl systems, ¹⁸F-fluoride nucleophilic displacement from an aryl diazonium salt, aryl nitro compound or an aryl quaternary ammonium salt are suitable routes to aryl-¹⁸F derivatives.

A ¹⁸F-labelled compound of the invention may be obtained by formation of ¹⁸F fluorodialkylamines and subsequent amide formation when the ¹⁸F fluorodialkylamine is reacted with a precursor containing, e.g. chlorine, P(O)Ph₃ or an activated ester.

A further approach for radiofluorination, which is particularly suitable for radiofluorination of peptides, is described in WO 03/080544 and uses thiol coupling. A precursor compound comprising one of the following substituents:

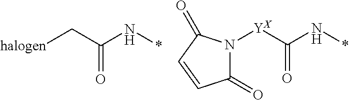

is reacted with a compound of Formula IV:

¹⁸F—X^{x}—SH (IV)

wherein Y^{X} is a linker moiety of formula -(L^{Y})_{y}- wherein L^{Y} is as previously defined for L, y is 1-10 and optionally includes 1-6 heteroatoms;
X^{x} is a linker of formula -(L^{x})ₓ- wherein L^{x} is as previously defined for L, x is 1-30 and optionally includes 1 to 10 heteroatoms; and,
* defines the point of attachment to the rest of the in vivo imaging agent;
to give radiofluorinated in vivo imaging agents of formula (IVa) or (IVb) respectively:

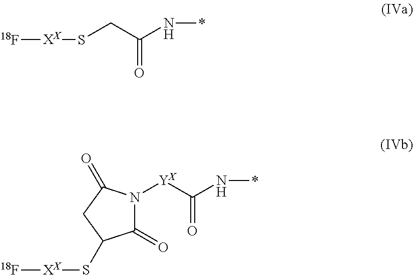

wherein X^{x}, Y^{x}, and * are as defined above.

An additional approach particularly suitable for radiofluorination of peptides is described in Poethko et al (J. Nuc. Med., 2004; 45: 892-902), and makes use of aminoxy coupling. Radiofluorination is carried out by reaction of a precursor compound of formula (V) with a compound of formula (Va):

R¹—* (V)

¹⁸F—X^{XI}—R² (Va)

or,
by reaction of a precursor compound of formula (VI) with a compound of formula (VIa)

R³—* (VI)

¹⁸F—X^{XII}—R⁴ (VIa)

wherein;
X^{XI} and X^{XII} are linker groups -(L^{XI})_{z}- wherein L^{XI} is as previously defined for L, z is 1-10 and optionally includes 1-6 heteroatoms;
R¹ is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;
R² is a functional group which, under mild conditions such as aqueous buffer, reacts site-specifically with R¹ yielding a stable conjugate. R² can be ammonia derivatives such as primary amine, secondary amine, hydroxylamine, hydrazine, hydrazide, aminoxy, phenylhydrazine, semicarbazide, or thiosemicarbazide, and is preferably a hydrazine, hydrazide or aminoxy group;
R³ is a functional group which reacts site-specifically with R⁴. R³ can be ammonia derivatives such as primary amine, secondary amine, hydroxylamine, hydrazine, hydrazide, aminoxy, phenylhydrazine, semicarbazide, or thiosemicarbazide, and is preferably a hydrazine, hydrazide or aminoxy group;
R⁴ is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;
to give a conjugate of formula (VII) or (VIII), respectively:

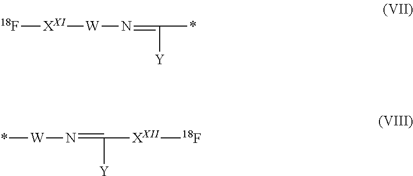

wherein W is —CO—NH—, —NH—, —O—, —NHCONH—, or —NHCSNH—, and is preferably —CO—NH—, —NH— or —O—; Y is H, C₁₋₆ alkyl or C₅₋₆ aryl substituents, and wherein X^{XI}, X^{XII} and * are as previously defined.

A preferred ¹⁸F labelled in vivo imaging agent of the invention is disclosed by Indrevoll et al (Bioorg. Med. Chem. Lett. 2006; 16: 6190-3). Further details of synthetic routes to ¹⁸F-labelled derivatives are described by Bolton (J. Lab. Comp. Radiopharm. 2002; 45: 485-528).

Preferred in vivo imaging moieties are those which can be detected externally in a non-invasive manner following administration in vivo, such as by means of SPECT, PET and MRI. Most preferred in vivo imaging moieties are radioactive, especially radioactive metal ions, gamma-emitting radioactive halogens and positron-emitting radioactive non-metals, particularly those suitable for imaging using SPECT or PET, e.g. ^{99m}Tc, ¹²³I, ¹¹C and ¹⁸F.

In a preferred embodiment, W₁ represents a linker moiety, Z₁ comprises an in vivo imaging moiety, W₂ represents an optional linker moiety, and Z₂ is hydrogen, and include the following:

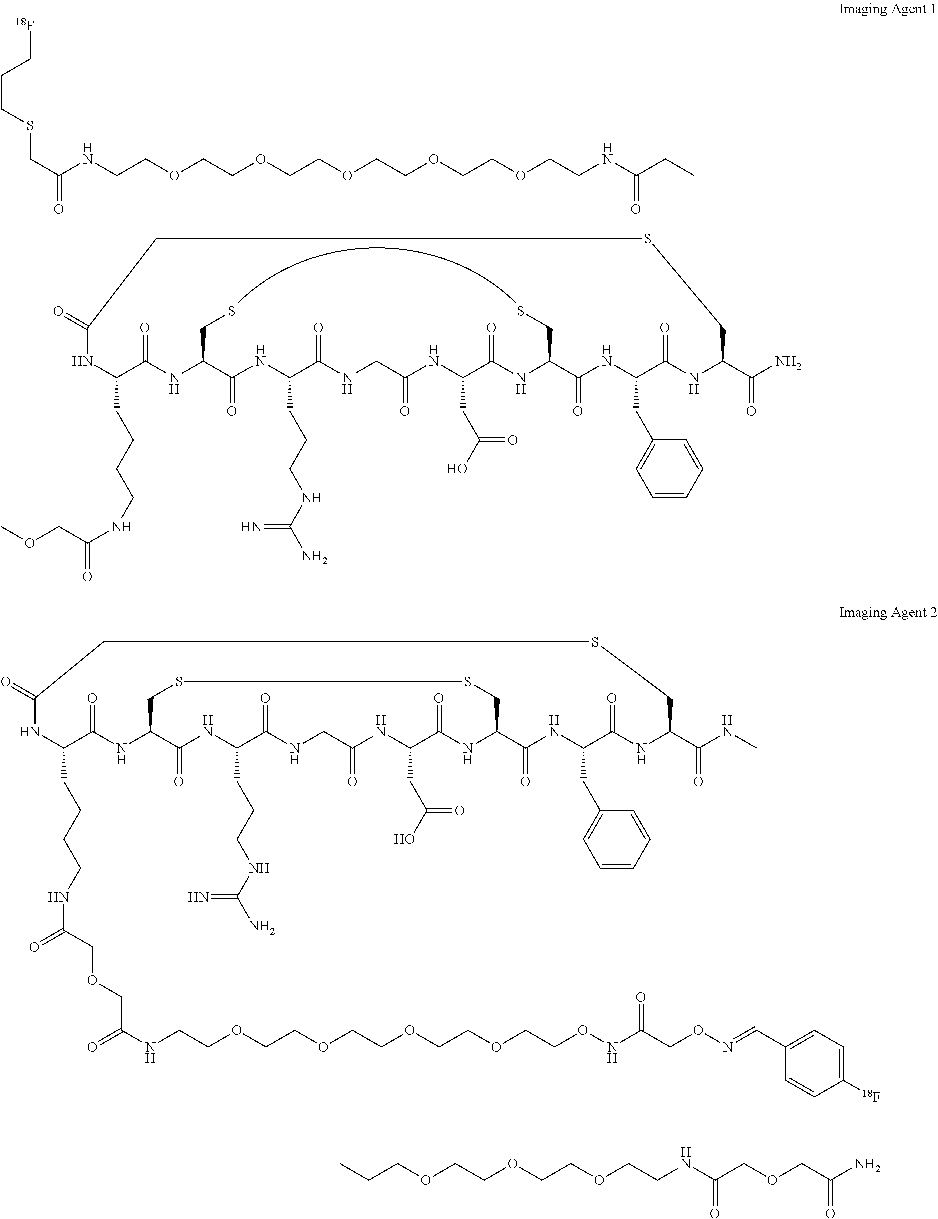

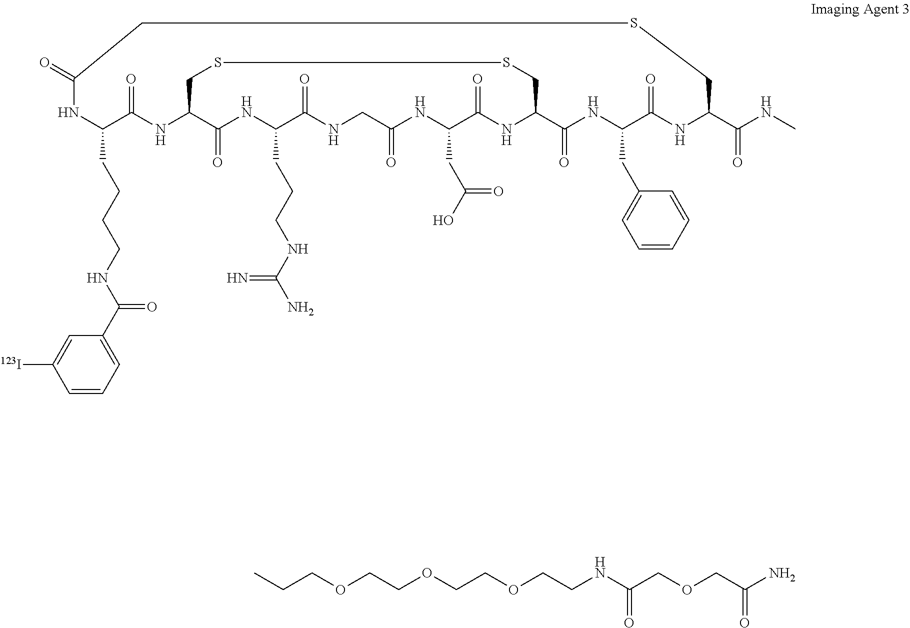

Methods for the preparation of Imaging Agents 1 and 3 are detailed in WO 2005/012335, and for the preparation of Imaging Agent 2 in Kenny et al (J. Nuc. Med. 2008; 49: 879-86).

In a yet further alternative preferred embodiment, W₁ represents an optional linker moiety, Z₁ is hydrogen, W₂ represents a linker moiety, and Z₂ comprises an in vivo imaging moiety, for example:

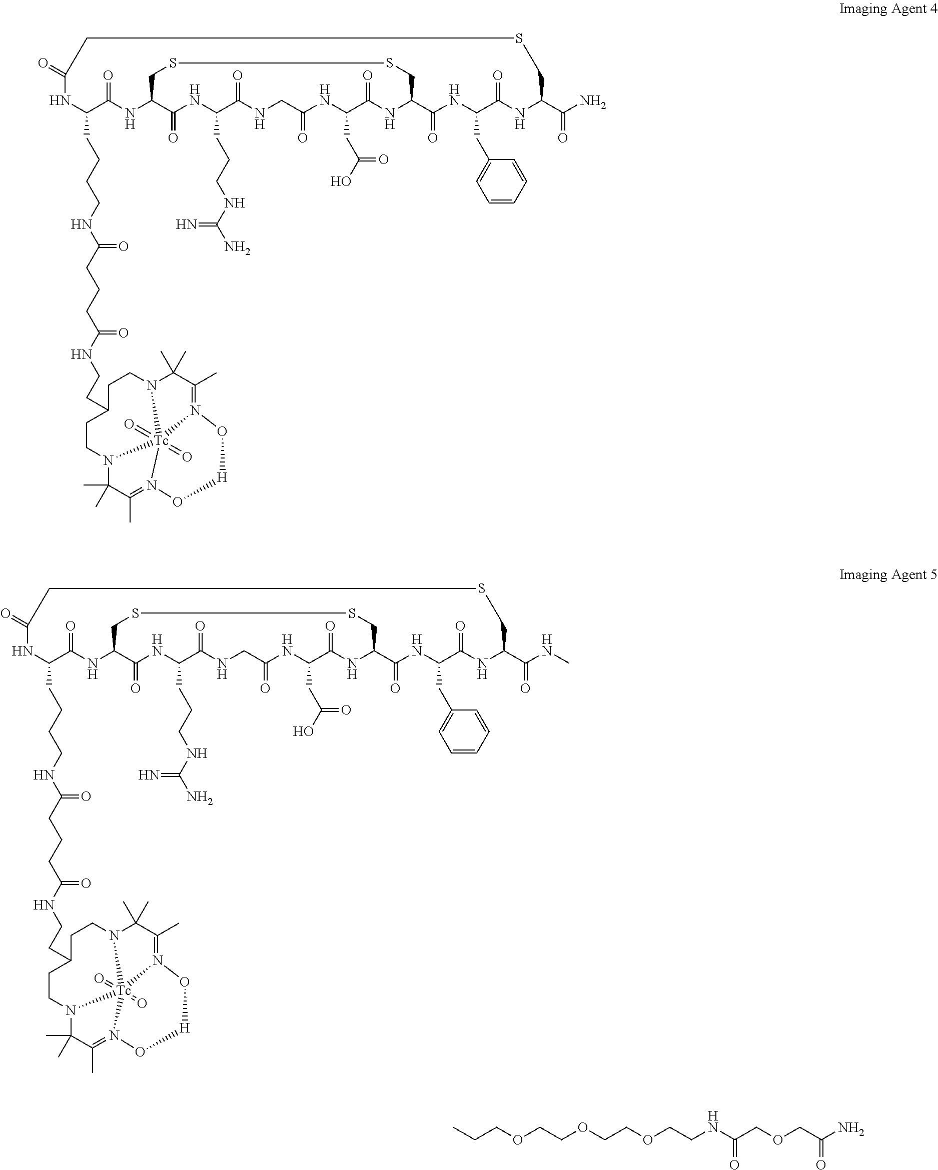

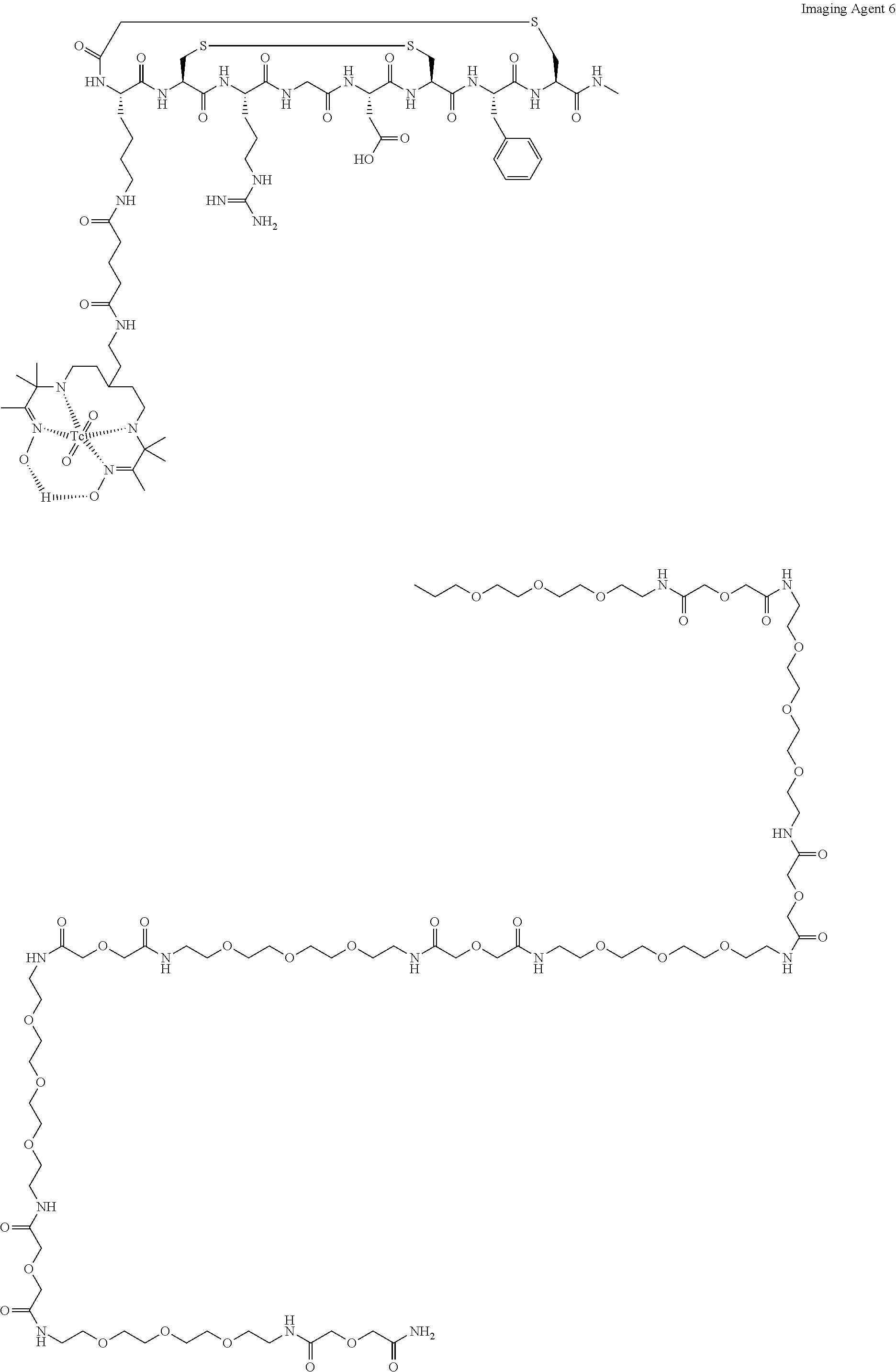

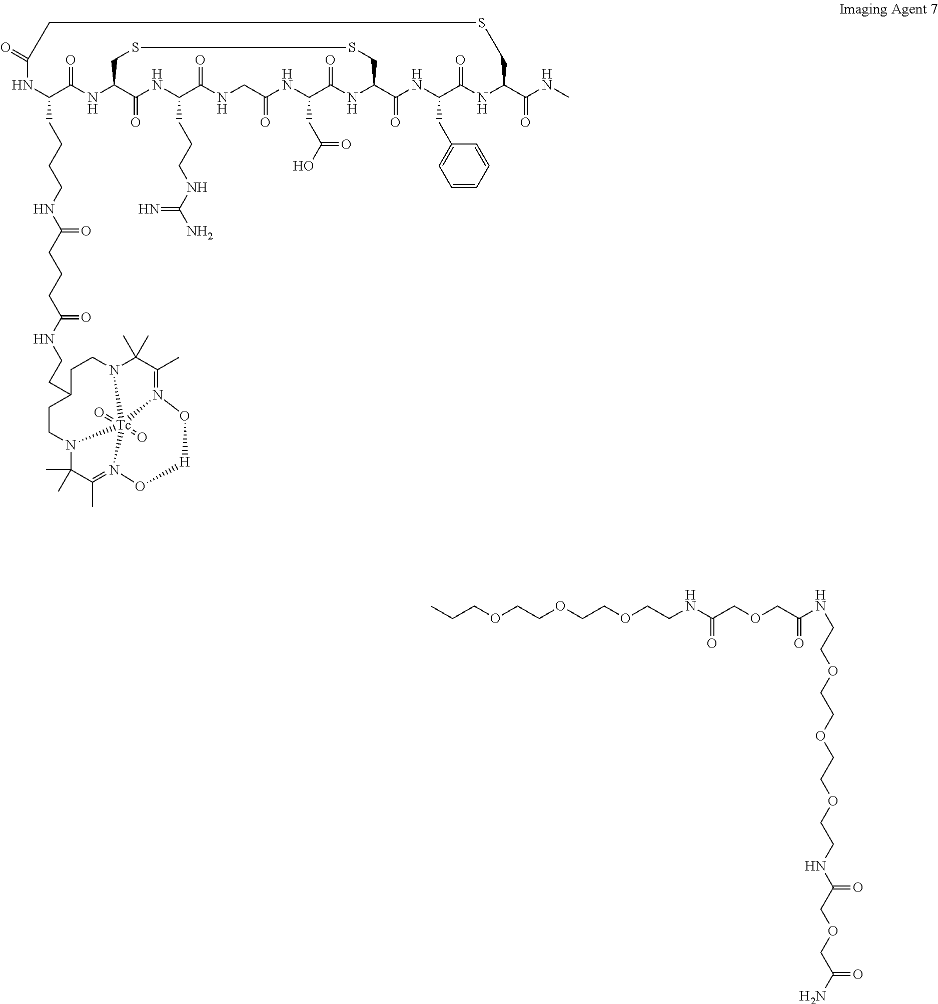

Methods for the preparation of Imaging Agents 4-7 are detailed in WO 2003/006491.

When the subject of the invention is an intact mammalian body in vivo, the in vivo imaging agent is preferably administered as a pharmaceutical composition. The broad definition of a pharmaceutical composition provided earlier in the specification applies. However, in this embodiment, the active agent is said in vivo imaging agent. The biocompatible carrier is a fluid, especially a liquid, in which the in vivo imaging agent is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such in vivo imaging agent pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can be withdrawn into clinical grade syringes at various time intervals during the viable lifetime of the preparation to suit the clinical situation.

Pre-filled syringes are designed to contain a single human dose, or “unit dose”, and are therefore preferably a disposable or other syringe suitable for clinical use. Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition may be prepared from a kit. Alternatively, it may be prepared under aseptic manufacture conditions to give the desired sterile product. The pharmaceutical composition may also be prepared under non-sterile conditions, followed by terminal sterilisation using e.g. gamma-irradiation, autoclaving, dry heat or chemical treatment (e.g. with ethylene oxide).

The present inventors propose that the method of the present invention would be useful to determine the optimal dose and regimen of inhibitors being tested in clinical studies comprising subjects suffering from melanoma. Another application is in the assessment of patient early response to these inhibitors. In addition, the method of the invention may be used to select those subjects most likely to respond to the inhibitor, e.g. as part of a registration trial only including patients most likely to respond. Should any of these inhibitors be approved for clinical use, the method of the invention could then be applied to enable decisions about whether to continue treatment with the inhibitors, alter the dose, or to pursue a different treatment strategy. Each of these uses forms a preferred aspect of the method of the invention.

In an alternative aspect, the present invention provides for use of an in vivo imaging agent that binds to αᵥβ₃ integrin in the method of the invention. For this aspect of the invention, the suitable and preferred embodiment of said in vivo imaging agent and said method are as defined above for said method of the invention.

In another alternative aspect, the present invention provides for use of an in vivo imaging agent that binds to αᵥβ₃ integrin in the manufacture of a medicament suitable for use in the method of the invention. For this aspect of the invention, the suitable and preferred embodiment of said in vivo imaging agent and said method are as defined above for said method of the invention.

The present invention furthermore provides a computer program product for use in carrying out the method and uses of the invention as described herein. For this aspect of the invention, the suitable and preferred embodiment of said in vivo imaging agent and said method are as defined above for said method of the invention.

The experimental example below demonstrates specific uptake of an αᵥβ₃ integrin-binding imaging agent into melanoma lesions.

### BRIEF DESCRIPTION OF THE EXAMPLES

Example 1 presents an in vivo imaging experiment where Imaging Agent 2 was used to image a human subject with confirmed metastatic melanoma.

### EXAMPLES

### Abbreviations Used in the Examples

MBq megabequerel(s)
PET positron emission tomography
CT computed tomography

### Example 1

### In Vivo Imaging Metastatic Melanoma

In vivo imaging using Imaging Agent 2 (having a binding affinity (IC₅₀) to αᵥβ₃ integrin of 11.1 nM; see Kenny et al J. Nuc. Med. 2008; 49: 879-86) was carried out on a 60-year-old female patient with biopsy confirmed metastatic melanoma.

365 MBq of Imaging Agent 2 (injectate prepared by the method described by Kenny et al, J. Nuc. Med. 2008; 49: 879-86) was administered to the patient. In vivo imaging data was acquired on a GE Discovery Rx PET/CT (GE Healthcare).

The first bed position of the whole body acquisition started at 41 minutes post-injection of Imaging Agent 2. The duration of each bed position was 5 minutes, and 5 bed positions were acquired with a 9 plane overlap. The data were corrected for deadtime, decay, randoms, scatter and attenuation, the latter using a energy scaled CT acquisition, and reconstructed with the Vue Point HD (GE Healthcare) reconstruction algorithm (8 iterations and 21 subsets).

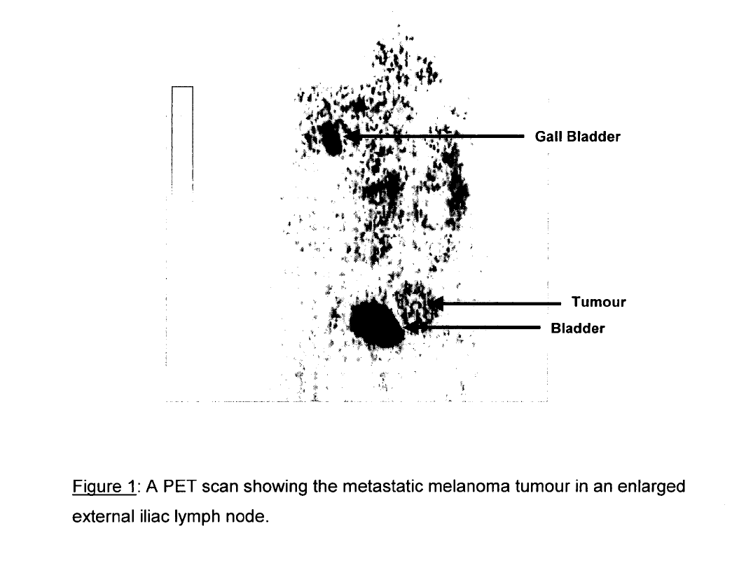

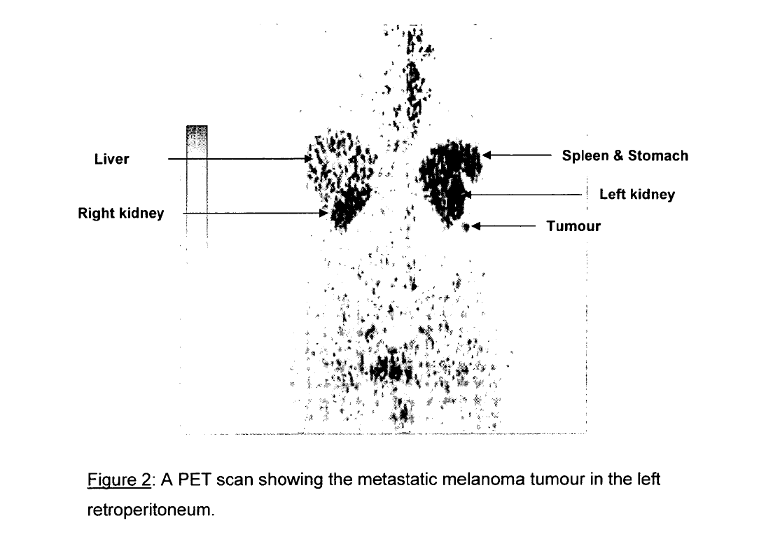

FIGS. 1 and 2 illustrate uptake of Imaging Agent 2 in confirmed metastasised melanoma lesions. This in vivo imaging procedure therefore demonstrates specific uptake of an αᵥβ₃ integrin-binding imaging agent into melanoma lesions.

What is claimed is:

1) A method for monitoring the effectiveness of an inhibitor, wherein said inhibitor is an inhibitor of B-raf, MEK1/2 (MEK: Mitogen-activated protein kinase/Extracellular signal related kinase Kinase), or ERK1/2 (ERK: Extracellular signal Related Kinase), said inhibitor being used to treat a subject suffering from melanoma, said method comprising:
(a) at a first point in time, carrying out an in vivo imaging procedure on said subject to generate a first in vivo image of a region of interest, wherein said region of interest comprises said melanoma, and wherein said in vivo imaging procedure comprises:
(i) administration to said subject of an in vivo imaging agent comprising a vector labelled with an in vivo imaging moiety, wherein said in vivo imaging agent binds to αᵥβ₃ integrin with a Ki of <10 nM in a competitive binding assay for αᵥβ₃ integrin where the Ki value is determined by competition with echistatin;
(ii) allowing the administered in vivo imaging agent of step (i) to bind to αᵥβ₃ integrin expressed by said melanoma;
(iii) detecting signals emitted by the in vivo imaging moiety of the bound in vivo imaging agent of step (ii); and,
(iv) converting the signals detected in step (iii) into a first in vivo image representative of αᵥβ₃ integrin expression on the surface of said melanoma cells in said region of interest;
(b) treating said subject with said inhibitor;
(c) at a second point in time, repeating the in vivo imaging procedure as defined in step (a) to generate a second in vivo image of said region of interest; and,
comparing said first in vivo image with said second in vivo image, whereby a decrease in said detected signals at said second point in time indicates effectiveness of said inhibitor in treating said melanoma.

2) The method of claim 1 wherein said melanoma comprises a mutation in the B-raf gene.

3) The method of claim 2 wherein said mutation is caused by the single substitution V599E.

4) The method of claim 1 wherein said subject is an intact mammalian subject in vivo.

5) The method of claim 4 wherein said subject is a human subject.

6) The method of claim 1 wherein said in vivo imaging agent is of Formula I: 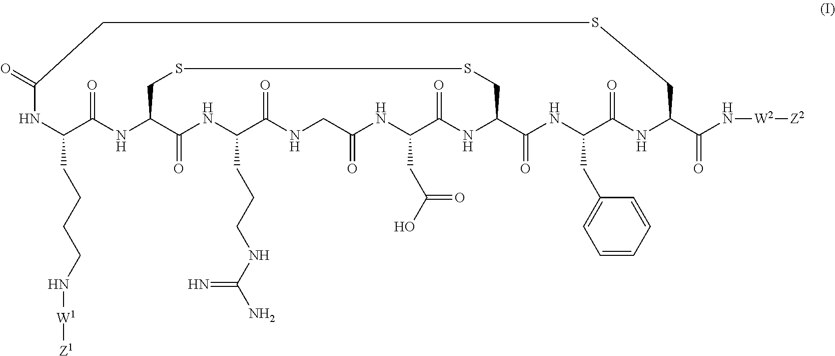
wherein:
W₁ and W₂ are independently an optional linker moiety, wherein said linker moiety is a bivalent radical of Formula -(L)ₙ- wherein:
each L is independently —C(═O)—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₄₋₈ cycloheteroalkylene group, a C₄₋₈ cycloalkylene group, a C₅₋₁₂ arylene group, a C₃₋₁₂ heteroarylene group, an amino acid, a polyalkyleneglycol, polylactic acid or polyglycolic acid moiety;
n is an integer of value 1 to 15;
and wherein each R′ group is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, or 2 or more R′ groups, together with the atoms to which they are attached form a carbocyclic, heterocyclic, saturated or unsaturated ring; and,
Z₁ and Z₂ are independently (i) a group comprising an in vivo imaging moiety, (ii) a sugar moiety, or (iii) hydrogen, with the proviso that at least one of Z₁ and Z₂ is an in vivo imaging moiety.

7) The method of claim 1 wherein said in vivo imaging moiety is selected from:
(a) a radioactive metal ion;
(b) a gamma-emitting radioactive halogen;
(c) a positron-emitting radioactive non-metal; and,
(d) a paramagnetic metal ion.

8) The method of claim 1 for use in clinical trials to determine the optimal dose and regimen of an inhibitor of B-Raf, MEK1/2 or ERK1/2 in the treatment of melanoma.

9) The method of claim 1 in the assessment of patient early response to an inhibitor of B-Raf, MEK1/2 or ERK1/2 in the treatment of melanoma.

10) The method of claim 1 in the selection of patients most likely to respond to an inhibitor of B-Raf, MEK1/2 or ERK1/2 in the treatment of melanoma.

11) The method of claim 1 for use in making a decision about whether to continue treatment of melanoma with an inhibitor of B-Raf, MEK1/2 or ERK1/2.

12. (canceled)

13. (canceled)

14) A computer program product for use in carrying out the method of claim 1.
